(19) United States
(12) Patent Application Publication
(10) Pub. No.: US 2021/0339164 A1
(43) Pub. Date: Nov. 4, 2021

(54) LIQUID PRODUCTION METHOD AND LIQUID PRODUCTION DEVICE

(71) Applicants: Shogo TORII, Shizuoka (JP); Shogo SUZUKI, Kanagawa (JP)

(72) Inventors: Shogo TORII, Shizuoka (JP); Shogo SUZUKI, Kanagawa (JP)

(21) Appl. No.: 17/255,828

(22) PCT Filed: Jun. 26, 2019
(86) PCT No.: PCT/JP2019/025453

§ 371 Date: Dec. 23, 2020

(30) Foreign Application Priority Data
Jun. 29, 2018 (JP) 2018-125170
Sep. 4, 2018 (JP) 2018-165408

(51) Int. Cl. B01D 11/02; C09B 61/00; C07C 33/02; C07D 311/04; C07D 307/62; C07D 493/08; C07C 13/20; C07C 39/00

(52) U.S. Cl. CPC B01D 11/0292; C09B 61/00; C07C 33/02; C07D 311/04; C07D 307/62; B01D 11/0288; C07C 13/20; C07C 39/00; B01D 11/0203; B01D 11/028; C07D 493/08

(57) ABSTRACT

Disclosed is a liquid production method and device for producing a liquid containing water-soluble components. The liquid production method includes extracting water-soluble components from a biological tissue, using a liquefied gas, to yield an extraction liquid, and vaporizing the liquefied gas from the extraction liquid obtained in the extracting to concentrate the extraction liquid.

### TECHNICAL FIELD

The disclosures discussed herein relate to a liquid production method and a liquid production device for producing a liquid containing water and water-soluble components.

### BACKGROUND ART

Essential oils are volatile oils that contain aromatic compounds, and are extracted from plants by steam distillation, solvent extraction, etc. (see, e.g., Patent Document 1).

### CITATION LIST

### Patent Literature

[PTL 1] Japanese Unexamined Patent Application Publication No. 2016-074820

### SUMMARY OF INVENTION

### Technical Problem

However, the steam distillation or solvent extraction requires heating for extracting or concentrating an extraction liquid, where the extracted components may be pyrolytically decomposed or proteins may be thermally denatured.

The object of the present disclosure is to provide a liquid production method and a liquid production device for extracting and concentrating water-soluble components contained in biological tissue within a predetermined temperature range, at which pyrolysis of the extracted components or thermal denaturation of proteins is unlikely to occur.

### Solution to Problem

According to one aspect of the present invention, there is provision of a liquid production method for producing a liquid containing water-soluble components, the liquid production method comprising:

extracting water-soluble components from a biological tissue, using a liquefied gas, to yield an extraction liquid; and

vaporizing the liquefied gas from the extraction liquid obtained in the extracting to concentrate the extraction liquid.

According to another aspect of the present invention, there is provision of a liquid production device for producing a liquid containing water-soluble components, the liquid production device comprising:

an extracting unit configured to extract water-soluble components from a biological tissue, using a liquefied gas, to yield an extraction liquid; and

a concentration unit configured to vaporize the liquefied gas from the extraction liquid obtained by the extracting unit to concentrate the extraction liquid.

### Advantageous Effects of Invention

According to aspects of the present invention, it is possible to provide a liquid production method and a liquid production device for producing a liquid, which are capable of extracting water-soluble components contained in biological tissue to yield an extraction liquid and concentrating the extraction liquid within a predetermined temperature, at which pyrolysis of the extracted components or thermal denaturation of proteins is unlikely to occur.

### BRIEF DESCRIPTION OF DRAWINGS

FIG. 1 is a flowchart illustrating an example of a liquid production method according to an embodiment;

FIG. 2 is a schematic diagram illustrating an example of a liquid production device according to an embodiment; and

FIG. 3 is a schematic diagram illustrating an example of a liquid production device in examples.

### DESCRIPTION OF EMBODIMENTS

In the following, embodiments of the present invention will be described with reference to the accompanying drawings.

Liquid Production Method

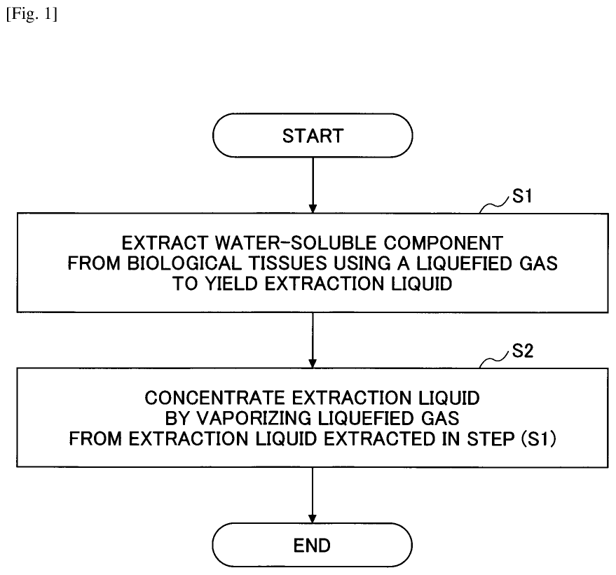

FIG. 1 is a flowchart illustrating an example of a liquid production method according to an embodiment.

The liquid production method includes (S1) extracting water-soluble components from a biological tissue, using a liquefied gas, to yield an extraction liquid, and (S2) concentrating the extraction liquid by vaporizing the liquefied gas from the extraction liquid obtained in step (S1). This liquid production method enables extraction and concentration of water-soluble components contained in a biological tissue within a predetermined temperature, at which pyrolysis of the extracted component or thermal denaturation of proteins is unlikely to occur, thereby producing a liquid containing water-soluble components.

In step (S1), for example, water-soluble components are extracted by bringing a liquefied gas into contact with a biological tissue. This improves mold resistance of a liquid containing the water-soluble components.

The liquefied gas can also dissolve cell membrane components to destroy cells of biological tissue.

In the specification and claims of the present application, a liquefied gas is defined as a liquefied product of a substance, which becomes a gas at normal temperature and pressure (0 oC, and 1 atm (0.101325 MPa)).

Examples of such a liquefied gas include, but are not limited to, dimethyl ether, ethyl methyl ether, formaldehyde, ketene, acetaldehyde, propane, butane, liquefied petroleum gas, and the like; and two or more types of liquefied gases may be used in combination. Of these, ethyl methyl ether and dimethyl ether are preferred, and dimethyl ether is particularly preferred, in view of being liquefied at relatively low temperature and low pressure.

Since dimethyl ether is liquefied at a temperature range of approximately 1 to 40 oC and a pressure range of 0.2 to 5 MPa, the cost of a liquid production device may be low. With the temperature of dimethyl ether being within a range of 1 to 40 oC, pyrolysis of water-soluble components contained in biological tissue or thermal denaturation of proteins is unlikely to occur. Since liquefied dimethyl ether readily vaporizes at normal temperature and pressure, liquefied dimethyl ether does not appreciably remain in a liquid that contains biological tissue-derived water-soluble components. Accordingly, use of liquefied dimethyl ether facilitates concentration of the extraction liquid while inhibiting pyrolysis of water-soluble components contained in biological tissue and thermal denaturation of proteins.

Examples of water-soluble components include, but are not limited to, aromatic compounds, natural pigment compounds, antioxidant compounds, antimicrobial compounds, antiviral compounds, and the like; and two or more types may be used in combination.

Step (S1) is conducted under an environment of a saturated vapor pressure or higher, such as inside of a closed extraction tank, in order to maintain a liquid state of the liquefied gas.

Any methods may be used to bring a liquefied gas into contact with biological tissue such as, but not limited to, immersion of biological tissue in a liquefied gas.

Preferably, a temperature of a liquefied gas is in a range of 1 to 80 oC, and more preferably in a range of 4 to 37 oC. The temperature of a liquefied gas being lower than 4 oC may damage the extracted components due to freezing of water, and the temperature of a liquefied gas being higher than 37 oC may cause pyrolysis of the extracted components and thermal denaturation of proteins.

In step (S2), for example, an extraction liquid extracted with a liquefied gas is returned to an environment of normal temperature and pressure.

Since a liquefied gas readily vaporizes at normal temperature and pressure, the liquefied gas may be vaporized from the extraction liquid to easily concentrate the extraction liquid.

Note that steps (S1) and (S2) may be repeated multiple times.

The liquid production method may further include a step (S3) of separating non-water-soluble components from the concentrated extraction liquid if containing the non-water-soluble components.

Here, since non-water-soluble components are soluble in a liquefied gas and are insoluble in water, non-water-soluble components precipitate when the liquefied dimethyl ether is vaporized.

In step (S3), for example, the concentrated extraction liquid may be filtered to separate the non-water-soluble components from the extraction liquid.

Biological Tissue

Examples of biological tissue include, but are not limited to, plant tissue including leaves, branches, trees, flower petals, stems, roots, pulp, carpels, seeds, etc.; soft tissue including human-derived or different mammalian-derived or skin, blood vessels, heart valves, cornea, amnion, dura, etc., or portions thereof; organs including heart, kidney, liver, pancreas, brain, etc., or portions thereof; and animal tissue such as bone, cartilage, tendon, or portions thereof.

Liquid Production Device

The liquid production device of the present embodiment is not particularly specified; the liquid production device may be any device that can extract water-soluble components from biological tissue using a liquefied gas to obtain an extraction liquid, and that can vaporize the liquefied gas from the extraction liquid to concentrate the extraction liquid.

The following illustrates a case where liquefied dimethyl ether is used as a liquefied gas.

The liquid production device of the present embodiment extracts water-soluble components by bringing liquefied dimethyl ether, which is generated by setting dimethyl ether at a saturated vapor pressure or higher, into contact with biological tissue to destroy cells. The liquefied dimethyl ether is then vaporized by setting the extraction liquid at a pressure below the saturated vapor pressure to concentrate the extraction liquid.

The liquid production device of the present embodiment includes a liquid feed unit configured to transfer liquefied dimethyl ether from a storage unit described below, an extraction unit configured to bring the transferred liquefied dimethyl ether into contact with biological tissue to extract water-soluble components, and an extrusion unit configured to extrude the extraction liquid from the extraction unit. The liquid production device of the present embodiment further includes a concentration unit configured to vaporize the liquefied dimethyl ether from the extraction liquid by adjusting temperature and/or pressure to concentrate the extraction liquid, and a condensing unit configured to condense the vaporized dimethyl ether by adjusting a temperature and/or pressure. In addition, the liquid production device of the present embodiment includes a storage unit configured to store liquefied dimethyl ether, a supply unit configured to supply the liquefied dimethyl ether to the storage unit; and a detector configured to detect a temperature and a pressure of the liquefied dimethyl ether.

The liquid feed unit is not particularly specified; any liquid feed unit that can adjust a flow rate of liquefied dimethyl ether may be used. Examples of such a liquid feed unit include a liquid feed pump, a heat driver, and the like.

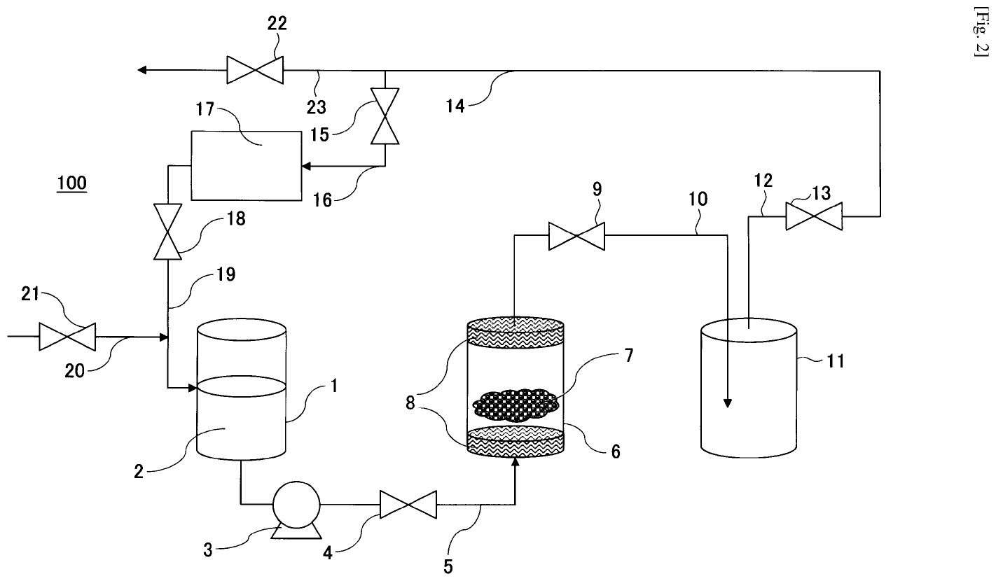

FIG. 2 illustrates an example of a liquid production device of the present embodiment.

The liquid production device 100 includes a storage tank 1 configured to store liquefied dimethyl ether 2, an extraction tank 6 configured to bring a biological tissue 7 into contact with the liquefied dimethyl ether 2 to extract water-soluble components, a pump 3 configured to transfer the liquefied dimethyl ether 2 from the storage tank 1 to the extraction tank 6, and a concentration tank 11 configured to vaporize the liquefied dimethyl ether from the extraction liquid to concentrate the extraction liquid.

The liquefied dimethyl ether 2 stored in the storage tank 1 is in a liquid state of dimethyl ether generated by setting the dimethyl ether at a saturated vapor pressure or higher.

The liquid production device 100 includes conduits 5, 10, 12, 14, 16, 19, 20, and 23 configured to extrude or introduce the liquefied dimethyl ether 2, and valves 4, 9, 13, 15, 18, 21, and 22 configured to control the extrusion of and the introduction of liquefied dimethyl ether 2 by adjusting pressures inside the respective tanks. Pressures inside the extraction tank 6 and the concentration tank 11 may be adjusted to maintain a liquid state of the liquefied dimethyl ether 2.

In the liquid production device 100, a pump 3, a valve 4, and a conduit 5 act as a liquid feed unit configured to introduce liquefied dimethyl ether 2 from the storage tank 1 to the extraction tank 6. The extraction tank 6 acts as an extraction unit. The conduit 10 and the valve 9 act as an extrusion unit to extrude the liquefied dimethyl ether 2 from the extraction tank 6. In addition, the concentration tank 11 acts as a concentration unit. A condenser 17 connected to the conduit 16 acts as a condensing unit. The conduit 12 and the valve 13 connected to the concentration tank 11 act as a vaporizing unit. The storage tank 1 acts as a storage unit. The conduits 19 and 20 act as a supply unit.

The liquid production device 100 may further include thermometers and pressure gauges for detecting a temperature and a pressure in respective tanks, agitators for agitation in the respective tanks, and devices etc., for distributing an inert gas such as nitrogen into the tanks and the conduits so as to purge an active gas such as oxygen.

The following illustrates a liquid production method for producing a liquid containing water-soluble components, using a liquid production device 100.

First, a biological tissue 7 is introduced into the extraction tank 6 in which respective filters 8 are located upstream and downstream. At this point, valves 4, 9, 13, 15, 18, 21, and 22 are closed. Here, when the liquefied dimethyl ether 2 is not sufficiently stored in the storage tank 1, the valve 21 is opened, and after supplying the liquefied dimethyl ether 2 into the storage tank 1 through the conduit 20, the valve 21 is closed. When the valve 21 is opened, the valve 18 may be opened; and when the valve 21 is closed, the valve 18 may be closed.

Next, the valve 4 is opened, and the liquefied dimethyl ether 2 in the storage tank 1 is extruded by the pump 3, and is introduced into the extraction tank 6 through the conduit 5 until the liquefied dimethyl ether 2 contacts the biological tissue 7; subsequently, the valve 4 is closed.

As a result, the water-soluble components in the biological tissue 7 are dissolved in the liquefied dimethyl ether 2 to be extracted.

Then, the valves 4 and 9 are opened, and liquefied dimethyl ether 2 is extruded from the storage tank 1 by the pump 3, and is introduced into the extraction tank 6 through the conduit 5. Thus, the extraction liquid in the extraction tank 6 is introduced into the concentration tank 11 through the conduit 10. That is, when the liquefied dimethyl ether is introduced from the storage tank 1 into the extraction tank 6, the introduced liquefied dimethyl ether extrudes the extraction liquid from the extraction tank 6, and the extruded extraction liquid is then introduced into the concentration tank 11. As a result, the extraction liquid in the extraction tank 6 is replaced with liquefied dimethyl ether. When the extraction liquid in the extraction tank 6 is replaced with liquefied dimethyl ether, the solubility up to saturation solubility increases; that is, the extraction power increases, making it possible to extract the extraction liquid more efficiently. Meanwhile, since the respective filters 8 are located upstream and downstream of the extraction tank 6, the biological tissue 7 remains in the extraction tank 6. That is, the extraction liquid is separated from the biological tissue 7 by introducing the liquefied dimethyl ether into the extraction tank 6.

Note that the timing of opening the valves 4 and 9 is after a sufficient time has elapsed for extracting the water-soluble components from the biological tissue 7. In this case, the liquefied dimethyl ether 2 may be stirred while being in contact with the biological tissue 7.

Next, the valves 9, 13, and 22 are closed, and paths from the valve 4 to the valve 13 are set to a pressure below the saturated vapor pressure of dimethyl ether, such that the liquefied dimethyl ether 2 in these paths is vaporized and discharged from the conduit 23 via the conduit 14. The pump 3 may be used to discharge the dimethyl ether as required.

As described above, the liquefied dimethyl ether 2 vaporizes from the extraction liquid, leaving a liquid containing the extracted water-soluble components in the concentration tank 11. In addition, an extraction residue of the biological tissue 7 remains in the extraction tank 6.

As illustrated above, the case where the valve 22 is opened and the valve 15 is closed has been described. However, the valve 22 may be closed and the valve 15 may be opened. This configuration introduces the vaporized dimethyl ether through conduit 16 into a condenser 17. As a result, the dimethyl ether introduced into the condenser 17 condenses to yield liquefied dimethyl ether. In this case, the liquefied dimethyl ether produced is introduced into the storage tank 1 through the conduit 19 by opening the valve 18. Accordingly, the liquid dimethyl ether 2 may be introduced from the storage tank 1 to the extraction tank 6 by the pump 3 again to extract the water-soluble components contained in the biological tissue 7. As a result, the water-soluble components contained in the biological tissue 7 can be extracted with a small amount of the liquefied dimethyl ether without changing or adding the liquefied dimethyl ether.

Although the case where the liquefied dimethyl ether 2 in the storage tank 1 is extruded discontinuously has been described above, the liquefied dimethyl ether 2 in the storage tank 1 may be extruded continuously.

Specifically, valves 4 and 9 may be open, and liquefied dimethyl ether 2 in the storage tank 1 may be continuously introduced into the extraction tank 6 through the conduit 5, and the extraction liquid in the extraction tank 6 may be continuously dispensed into the concentration tank 11 through the conduit 10. In this case, it is preferable to construct an internal structure of the extraction tank 6 such that the liquefied dimethyl ether 2 can be in continuous contact with the biological tissue 7.

In the liquid production device 100, the gas/liquid state of the dimethyl ether is changed by changing a pressure inside a device. However, the gas/liquid state may be changed by changing a temperature instead of a pressure.

### EXAMPLES

While examples according to embodiments of the present invention are described below, the embodiments of the present invention is not limited to the examples.

### Example 1

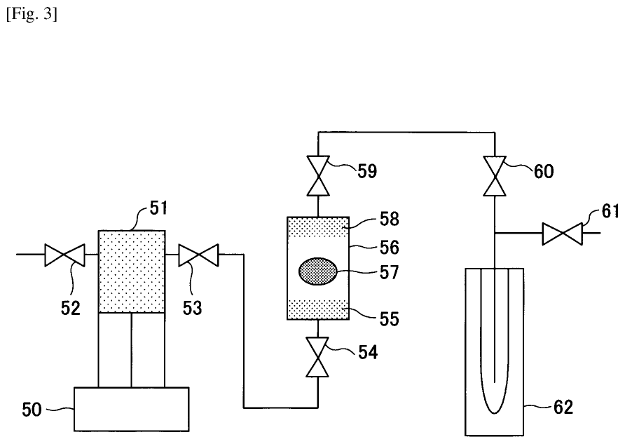

A liquid production device of FIG. 3 was used to prepare liquid containing water-soluble components from Kuromoji (Lindera umbellata).

Specifically, an extraction tank 56 having an internal volume of 25 mL, in which filters 55 and 58 were located upstream and downstream, was prepared, and the extraction traction tank 56 was charged with 6.0 g of Kuromoji (Lindera umbellata) with a water content of 10 wt %, where Kuromoji was pulverized to a length of approximately 1 mm or less as a biological tissue 57. Next, the valve 52 was opened and the valve 53 was closed, and the syringe pump 50 was filled with dimethyl ether to generate liquefied dimethyl ether 51 at 25 oC and 0.7 MPa. The content of the concentration tank 62 was then substituted with dimethyl ether in advance, and the valves 52, 53, 54, 59, 60, and 61 were closed. Next, valves 53, 54, 59, and 60 were opened and the liquefied dimethyl ether 51 was fed from the syringe pump 50 to the extraction tank 56. When the extraction tank 56 was filled with liquefied dimethyl ether, the syringe pump 50 was stopped, the valves 54 and 59 were closed, and the biological tissue 57 was immersed in the liquefied dimethyl ether. Next, the valves 54 and 59 were opened, liquefied dimethyl ether 51 was fed from the syringe pump 50 to the extraction tank 56, and 60 mL of the extraction liquid was collected in the concentration tank 62. In this case, the flow rate of the liquefied dimethyl ether 51 was adjusted to 2.5 mL/min, and the residence time of the liquefied dimethyl ether in the extraction tank 56 was set at 10 minutes. Next, after the valve 60 was closed and the concentration tank 62 was removed from the device, liquefied dimethyl ether was vaporized from the extraction liquid collected in the concentration tank 62 within a fume hood with a room temperature and ambient pressure to obtain a concentration liquid containing water-soluble components. The observation indicated that non-water-soluble components were suspended in the concentration liquid.

After the above operations were repeated twice, the valve 54 was closed, the valves 59, 60, and 61 were opened, and the inside of the extraction tank 56 was set at an atmospheric pressure. The liquefied dimethyl ether in the extraction tank 56 was vaporized and discharged, and the remaining biological tissue 57 after the extraction was collected as an extraction residue.

The resulting concentration liquid was filtered through a filter with a pore size of 0.47 μcm to remove non-water-soluble components to yield 0.2 g of a liquid containing the water-soluble components.

When the liquid containing the water-soluble components was analyzed by gas chromatography and liquid chromatography, the water-soluble components included linalol, 1.8-cineol, and limonene as aromatic compounds, and polyphenols as antioxidants.

### Comparative Example 1

A liquid containing the water-soluble components was prepared from Kuromoji in the same manner as Example 1, except that a steam distillation method was used.

Specifically, 6.0 g of Kuromoji and 100 g of water were placed in a flask 1, only 300 g of water was placed in the flask 2, and the flask 2 was heated by a gas burner to generate water vapor. The water vapor was then blown into the flask 1 so that water vapor was contacted to the Kuromoji, and the water vapor was subsequently cooled to be condensed by a condenser; as a result, 70 g of a liquid containing water and water-soluble components was obtained. Furthermore, the liquid containing the water-soluble components was concentrated with heating to yield 10 g of a concentrated liquid containing the water-soluble components.

Mold Resistance

When a liquid containing the water-soluble components of Example 1 and a liquid containing the water-soluble components of Comparative Example 1 were refrigerated for 60 days, the liquid of Comparative Example 1 developed mold and became cloudy, while the liquid of Example 1 was clear without mold. This may be because the liquid of Example 1 was sterilized by liquefied dimethyl ether when extracting of the extraction liquid.

### Example 2

A liquid containing the water-soluble components was prepared in the same manner as Example 1, except that 15 g of rose petals with a water content of 80 wt % were used as the biological tissue 57, to yield 6.4 g of a liquid containing water-soluble components.

The water-soluble components contained natural pigment compounds derived from rose petals, so the liquid was red.

### Comparative Example 2

A liquid containing the water-soluble components was prepared from rose petals in the same manner as Example 2, except that the steam distillation method was used.

Specifically, 15.0 g of rose petals and 100 g of water were placed in a flask 1, only 300 g of water was placed in a flask 2, and the flask 2 was heated by a gas burner to generate water vapor. The water vapor was then blown into the flask 1 so that water vapor was contacted to the rose petals, and the water vapor was subsequently cooled to be condensed by a condenser; as a result, 70 g of a liquid containing water and water-soluble components was obtained. Furthermore, the liquid containing the water-soluble components was concentrated with heating to yield 10 g of a concentrated liquid containing the water-soluble components.

The water-soluble components did not contain natural pigment compounds derived from rose petals and were clear and colorless.

### Example 3

A liquid containing the water-soluble components was prepared in the same manner as Example 1, except that 15 g of grapeseed powder with a water content of 10 wt % was used as the biological tissue 57, to yield 5.0 g of a liquid containing water-soluble components.

The result of analyzing the liquid containing water-soluble components by liquid chromatography indicated that the water-soluble components included a polyphenol as an antioxidant compound, and catechin as an antimicrobial compound and an antiviral compound.

### Comparative Example 3

A liquid containing the water-soluble components was prepared from grapeseed powder in the same manner as Example 3, except that the steam distillation method was used.

Specifically, 15.0 g of grapeseed powder and 100 g of water were placed in a flask 1, only 300 g of water was placed in a flask 2, and the flask 2 was heated by a gas burner to generate water vapor. The water vapor was then blown into the flask 1 so that water vapor was contacted to the grapeseed powder, and the water vapor was subsequently cooled to be condensed by a condenser; as a result, 70 g of a liquid containing water and water-soluble components was obtained. Furthermore, the liquid containing the water-soluble components was concentrated with heating to yield 10 g of a concentrated liquid containing the water-soluble components.

The result of analyzing the liquid containing water-soluble components by liquid chromatography indicated that the water-soluble components included a polyphenol as an antioxidant compound, and catechin as an antimicrobial compound, and an antiviral compound.

### Example 4

A liquid containing the water-soluble components was prepared in the same manner as Example 1, except that 10 g of pig liver with a water content of 70 wt % was used as the biological tissue 57, to yield 5.0 g of a liquid containing water-soluble components.

The result of analyzing the liquid containing water-soluble components by liquid chromatography indicated that the water-soluble components contained an ascorbic acid (vitamin C) as an antioxidant compound.

### Comparative Example 4

A liquid containing the water-soluble components was prepared from pig liver in the same manner as Example 4, except that the steam distillation method was used.

Specifically, 10.0 g of pig liver and 100 g of water were placed in a flask 1, only 300 g of water was placed in a flask 2, and the flask 2 was heated by a gas burner to generate water vapor. The water vapor was then blown into the flask 1 so that water vapor was contacted to the pig liver, and the water vapor was subsequently cooled to be condensed by a condenser; as a result, 70 g of a liquid containing water and water-soluble components was obtained. Furthermore, the liquid containing the water-soluble components was concentrated with heating to yield 10 g of a concentrated liquid containing the water-soluble components.

The result of analyzing the liquid containing water-soluble components by liquid chromatography indicated that the water-soluble components contained an ascorbic acid (vitamin C) as an antioxidant compound.

### REFERENCE SIGNS LIST

1 storage tank

2 liquefied dimethyl ether

3 pump

6 extraction tank

7 biological tissue

11 concentration tank

100 liquid production device

The present application is based on and claims the benefit of priority of Japanese Priority Application No. 2018-125170 filed on Jun. 29, 2018, and Japanese Priority Application No. 2018-165408 filed on Sep. 4, 2018, the entire contents of which are hereby incorporated herein by reference.

1. A liquid production method for producing a liquid containing water-soluble components, the liquid production method comprising:
extracting water-soluble components from a biological tissue, using a liquefied gas, to yield an extraction liquid; and
vaporizing the liquefied gas from the extraction liquid obtained in the extracting to concentrate the extraction liquid.

2. The liquid production method according to claim 1, further comprising:
vaporizing the liquefied gas to separate non-water-soluble components from the extraction liquid.

3. The liquid production method according to claim 1, wherein
the extracting is performed under a predetermined environment in which the liquefied gas maintains a liquid state.

4. The liquid production method according to claim 3, wherein
the liquefied gas is liquefied dimethyl ether, and
the predetermined environment is an environment with a temperature range of 1 to 40° C., and a pressure range of 0.2 to 5 MPa.

5. The liquid production method according to claim 1, wherein
the water-soluble components include one or more compounds selected from a group consisting of an aromatic compound, a natural pigment compound, an antioxidant compound, an antimicrobial compound, and an antiviral compound.

6. A liquid production device for producing a liquid containing water-soluble components, the liquid production device comprising:
an extracting unit configured to extract water-soluble components from a biological tissue, using a liquefied gas, to yield an extraction liquid; and
a concentration unit configured to vaporize the liquefied gas from the extraction liquid obtained by the extracting unit to concentrate the extraction liquid.
